(19) United States
(12) Patent Application Publication
(10) Pub. No.: US 2026/0359980 A1
(43) Pub. Date: Nov. 12, 2026

(54) SYSTEMS AND METHODS TO QUANTIFY UVEOSCLERAL OUTFLOW FROM OCULAR ULTRASOUND IMAGES

(71) Applicant: Ohio State Innovation Foundation, Columbus, OH (US)

(72) Inventors: Sayoko Moroi-Fetters, Columbus, OH (US); Yanhiu Ma, Columbus, OH (US); Jun Liu, Columbus, OH (US); Sunny Kwok, Columbus, OH (US); Ping Zhang, Columbus, OH (US); Biswajit Padhi, Columbus, OH (US)

(21) Appl. No.: 19/664,274

(22) Filed: Apr. 30, 2026

Related U.S. Application Data
(60) Provisional application No. 63/798,179, filed on May 1, 2025.

(51) Int. Cl. G06T 7/00; G06T 7/11; G16H 50/20

(52) U.S. Cl. CPC G06T 7/0012; G06T 7/11; G16H 50/20; G06T 2207/10132; G06T 2207/30041

(57) ABSTRACT

Disclosed herein are systems, methods and computer-program products for characterizing uveoscleral flow (Fu) from ultrasound biomicroscopy (UBM) by analyzing pixel intensity distributions in the anterior perilimbal sclera and ciliary body.

### CROSS REFERENCE TO RELATED APPLICATION

This application claims priority to and benefit of U.S. provisional patent application Ser. No. 63/798,179 filed May 1, 2025, which is fully incorporated by reference and made a part hereof.

### GOVERNMENT SUPPORT CLAUSE

This invention was made with government support under grant number R01 EY022124 awarded by the National Eye Institute (NEI) of the National Institute of Health (NIH). The government has certain rights in the invention.

### BACKGROUND

### Field of the Invention

Described herein are systems and methods to assess uveoscleral outflow in vivo. In some instances, the uveoscleral outflow may be used for determination of intraocular pressure (IOP).

### Background

IOP is an important physiological parameter for eye health to maintain the normal eye globe shape and for ocular nutrition. The normal physiological range of IOP is 10-21 mmHg (Allingham et al. 2020). Eye health can be compromised if the IOP is too low or too high.

If the IOP is very low, which is called hypotony, then eye health can suffer due to insufficient intraocular fluid, primarily aqueous humor, to maintain the shape of the globe and for nutrition of ocular structures. In the setting of hypotony, any of the following potential complications may be present in the eye: the clarity of the cornea is compromised by cornea edema or swelling of the cornea, irregularity of the cornea structure such as Descemet folds, shallow or flat anterior chamber, cataract progression, choroidal effusions, choroidal hemorrhage, retinal detachment, scleral infolding, and even phthisis. Any of the hypotony-related complications decreases vision and can potentially lead to irreversible blindness.

If the IOP is high, then the patient is at risk for glaucoma, which is a leading cause of irreversible blindness in the US and world (Bourne et al. 2016). Numerous clinical trials have demonstrated that IOP is a risk factor for glaucoma, and that effective lowering of IOP can slow vision loss from glaucoma (De Moraes, Liebmann, and Levin 2017; Ismail, Azuara-Blanco, and Ramsay 2014). Vision loss from glaucoma is due to damage of the optic nerve fibers, with associated retinal nerve fiber layer thinning, ganglion cell atrophy, and a characteristic feature of progressive ‘cupping’ of the optic disc due to loss of the nerve fibers. This damage to the optic nerve fibers is permanent, and can lead to blindness.

IOP is a dynamic physiological parameter that is determined by aqueous humor dynamic variable of fluid production, outflow through the trabecular meshwork, outflow through the uveoscleral path, and episcleral venous pressure. With the exception of uveoscleral outflow, all the other variables can be measured with specific instruments.

Emerging clinical evidence identifies large IOP fluctuation as a driver of glaucoma progression. Current practice is based on point-of-care IOP measured during office hours and this practice has limitations because most patients with elevated IOP do not develop glaucoma, and 50% or more of patients do not have elevated IOP at diagnosis. There remain knowledge gaps because point-of-care measurements fail to characterize dynamic IOP fluctuation, and there are no clinical risk factors for large IOP fluctuation.

Therefore, systems and methods are desired that overcome challenges to assess uveoscleral outflow, some of which are described herein. Specifically, systems and methods are desired to qualitatively assess uveoscleral outflow in vivo using ultrasound images, with the goal to quantitate uveoscleral outflow and using the measured uveoscleral outflow that determines IOP.

### SUMMARY

Disclosed and described herein are systems and methods for qualitative assessment of uveoscleral outflow in vivo using ultrasound images with the goal toward quantitative measure of uveoscleral outflow that determines IOP.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 illustrates probe placement for an exemplary ultrasound biomicroscopy (UBM) protocol;

FIGS. 2A-2G illustrate a process of intensity analysis use for UBM images;

FIG. 3 illustrates an exemplary intensity gradient plot along the central A-line where a dip in the curve indicates the uveoscleral outflow; and

FIG. 4 illustrates example computing device upon which embodiments of the invention may be implemented.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present methods and systems may be understood more readily by reference to the following detailed description of preferred embodiments and the Examples included therein and to the Figures and their previous and following description.

While IOP is a relatively simple measurement obtained during a clinic visit, IOP is a complex physiological trait with several variables that include aqueous humor flow (AHF), uveoscleral outflow (Fu), outflow facility (Ctrab) and episcleral venous pressure (Pe) (Toris, Gagrani, and Ghate 2021). These variables are illustrated in the modified Goldmann equation below:

$$IOP = \frac{AHF - Fu}{C t r a b} + P e$$

Variables that impact these physiological factors are shown in Table 1, below:

TABLE 1

| Physiological Factor | Variables |
|---|---|
| AHF (aqueous humor flow in | General (age; circadian rhythm; |
| microliters/min) | exercise) |
| | Disease (cardiovascular; uveitis; |
| | retinal/choroidal detachment) |
| | Treatment (pharmacologic and |
| | surgical) |
| Fu (uveoscleral outflow in | General (age) |
| microliters/min) | Disease (myotonic dystrophy and |
| | cyclodialysis) |
| | Treatment (pharmacologic) |
| Ctrab (outflow facility in | General (age) |
| microliters/min/mm Hg) | Disease (glaucoma and angle |
| | scarring) |
| | Treatment (pharmacologic and |
| | surgical) |
| Pe (episcleral venous pressure in | General (body position) |
| mm Hg) | Disease (thyroid disease; |
| | carotid/dural-cavernous sinus |
| | fistula; Sturge-Weber) |

These variables are assessed in clinical research called aqueous humor dynamic (AHD) studies. With the exception of uveoscleral flow (Fu), there are specific instruments to measure IOP by tonometry and ocular response analyzer, AHF by fluorophotometry, Ctrab by tonography, and Pe by episcleral venomanometry. There is no instrument to assess or measure Fu, so it is calculated using the modified Goldmann equation, above. Glaucoma medications lower IOP and are characterized by mechanism of action by drug target and mechanism of action by physiological variable determined by AHD studies.

Instead of using the Goldmann equation, the disclosed systems and methods characterize uveoscleral flow from ultrasound biomicroscopy (UBM) by analyzing pixel intensity distributions in the anterior perilimbal sclera(S) and ciliary body (CB). A S/CB intensity ratio is calculated as a structural biomarker, with automated extraction performed using annotated reference images. A stepped approach of human-in-the-loop annotations is used, then machine learning (ML) and/or deep learning (DL) are used to segment regions of interest (ROI) into peri-limbal sclera and ciliary body. DL is used in these ROIs to extract pixel intensity values, calculate intensity ratios (ciliary body/sclera), and perform correlation analyses and test associations with calculated uveoscleral outflow. Differential signals among the ROIs are used to indicate relative amounts of uveoscleral flow and will be associated with low versus large IOP fluctuation.

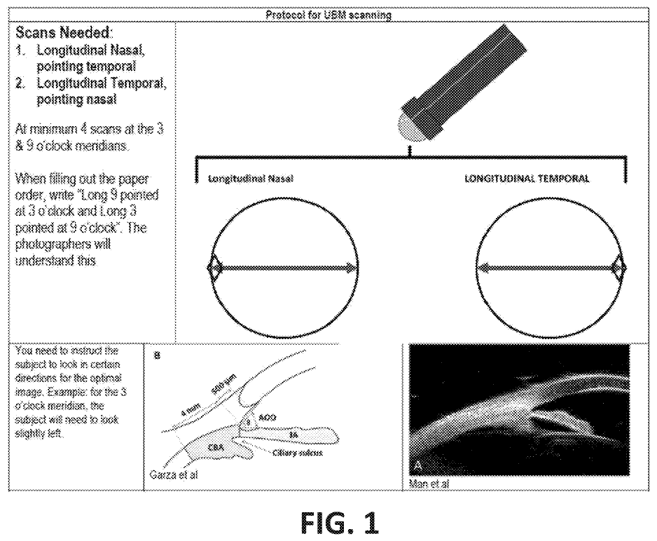

A UBM protocol acquires longitudinal images at nasal and temporal meridians, and additional meridians along the “clock hour” region of the eye as described later in this paragraph. For example, a high=frequency (e.g., 20 MHz., 50 MHz., etc.) probe (Quantel Medical or other industry ultrasound) may be used in supine position under topical anesthesia. The protocol involves placing a patient/subject in supine position and a topical anesthesia is applied. In some instances, an ocular gel may be applied on the ocular surface as coupling agent (this is optional, but may give better quality images rather than depending on just the tears on the ocular surface). Referring to FIG. 1, ultrasound scans are obtained at varying angles, for example the longitudinal nasal, pointing temporal; and longitudinal temporal, pointing nasal. For example, four scans may be obtained at the 3 and 9 o'clock meridians of the patient's eye, and then more scans along additional clock hours of the eye (varying angles). The scans are generally obtained in video mode.

The captured images are aligned for consistent measurement. For example, reference lines in the images may be use for alignment. For example, software executing an algorithm may receive the images and a computer executing a software algorithm detects any tilt in the angles of the reference marks and rotates the image to correct orientation. Both, a base image and an annotated image are rotated together to maintain alignment to achieve a standard vertical orientation across all images.

Using B-mode images, the anterior sclera and ciliary body are manually segmented relative to the limbus. Pixel intensity gradients along multiple vertical A-lines sampled evenly across the ROI are analyzed and averaged to delineate the intensity change at the transition band from sclera-to-potential subscleral space-to-ciliary body using custom algorithms. Specifically, two vertical lines marking the location of limbus and 4 mm posterior from limbus (toward the anterior sclera) are drawn as the boundaries for our region of interest (ROI). The interface between the anterior sclera and ciliary body is manually traced first. The anterior surface of the anterior sclera is then traced to connect the ROI boundaries and the anterior scleral-to-potential subscleral space-to-ciliary body interface for the segmentation of the anterior sclera. Similarly, the posterior surface of the ciliary body is traced to connect the ROI boundaries and the anterior scleral-to-potential subscleral space-to-ciliary body interface for the segmentation of the ciliary body. The average pixel intensity values within the ciliary body and anterior sclera regions are assessed, and the ratio between them (i.e., ciliary body/anterior sclera) is calculated. In general, the pixel intensity of the sclera is relatively high due to the compact nature of this tissue and relatively low aqueous humor content. In contrast, the pixel intensity of the ciliary body is more variable due to the nature of the complex tissues and the relative amount of aqueous humor in the tissue as part of the Fu pathway represented in the modified Goldmann equation. Preliminary data aligns with the hypothesis that relative differences in pixel intensity can be detected in the ROI under baseline conditions that demonstrate a trend with IOP with a higher signal intensity ratio indicating a higher IOP under baseline conditions. As part of the AHD protocol, IOP was measured using an Ocular Response Analyzer and also with several other tonometers.

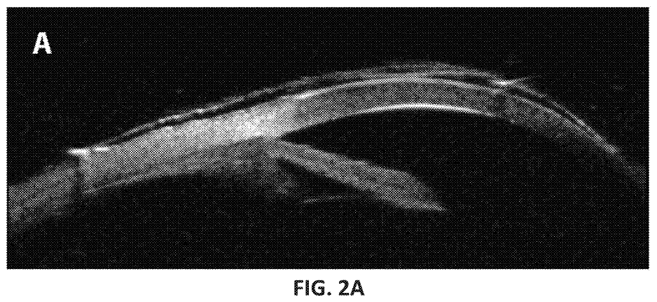

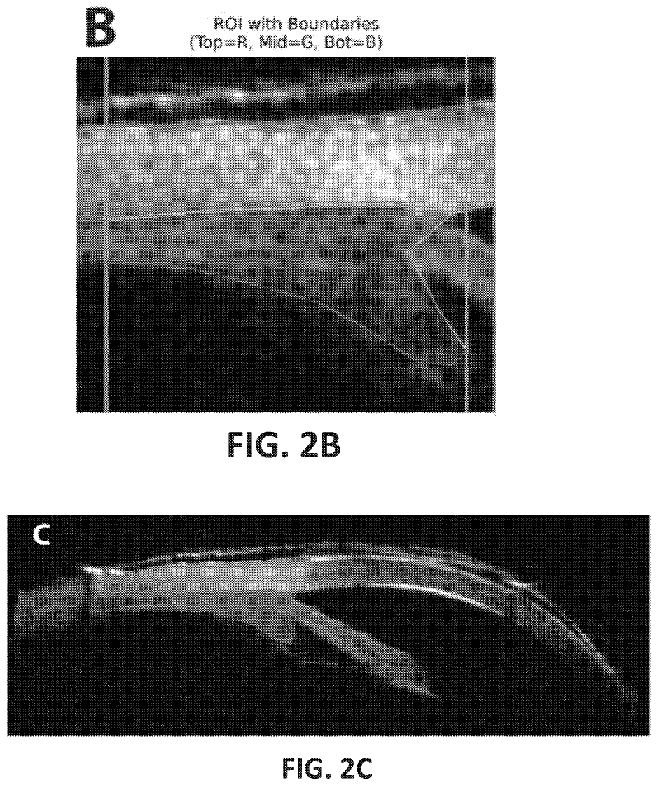

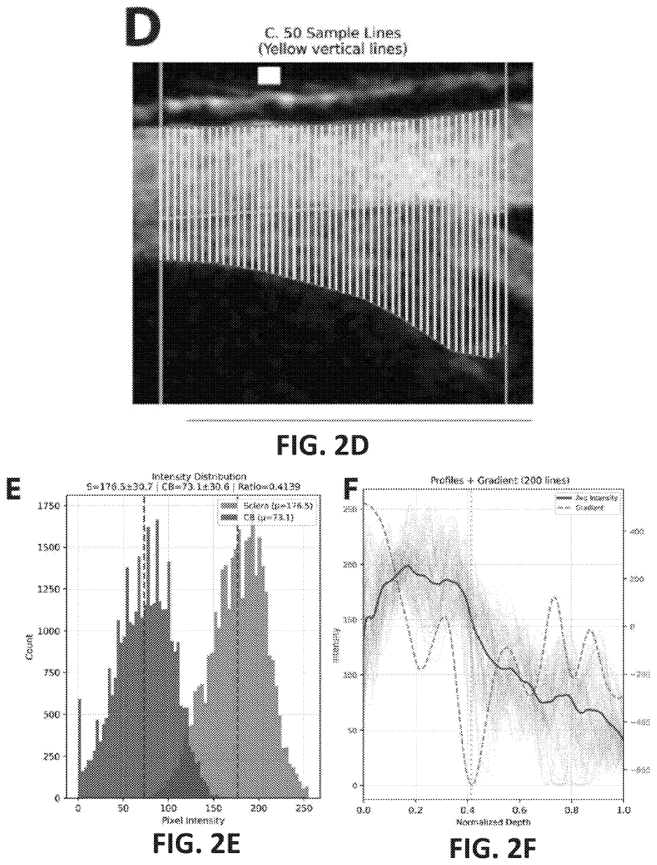

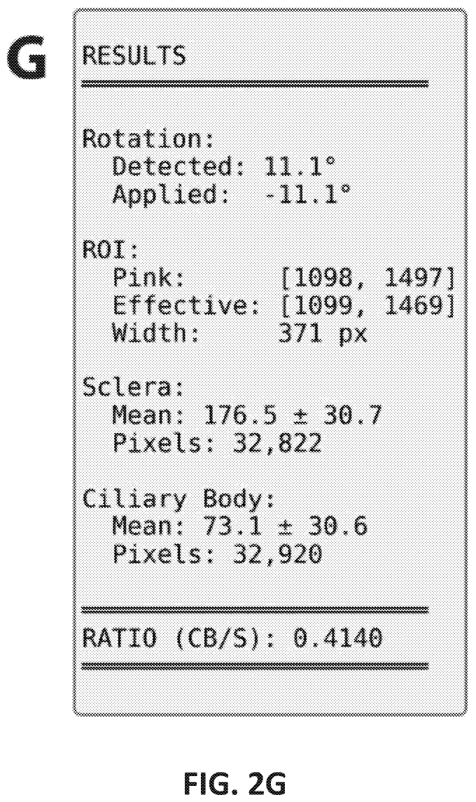

FIGS. 2A-G illustrate a process of intensity analysis use for UBM images. In FIG. 2A, an original longitudinal UBM image is acquired. In FIG. 2B, ROI tissue boundaries are identified: sclera top (red), sclera/CB junction (green), CB bottom (blue). In some instances, this may be performed in a human-in-the-loop process where a person identified and marks the ROIs. In FIG. 2C, a final segmentation mask is created with sclera (red) and ciliary body (blue) regions. FIG. 2D illustrates vertical sampling lines (n=50) for intensity profiling, across the region of interest to generate depth-wise intensity profiles. FIG. 2E shows pixel intensity distributions for sclera (red) and CB (blue) ROIs, includes all pixels within the masked regions, demonstrating clear separation between sclera (higher mean intensity: 176.5±30.7) and ciliary body (lower mean intensity: 73.1±30.6). FIG. 2F illustrates depth-normalized intensity profiles with individual profiles (grey), average (blue) and gradient (red dashed). And, FIG. 2G shows example summary results with CB/Sclera intensity ratio=0.414.

In one aspect, the stepped approach of human-in-the-loop annotations combined with statistical and image analysis techniques implemented in Python™ is used to segment regions of interest (ROI) into peri-limbal sclera and ciliary body regions. Image analysis and statistical methods are used in these ROIs to extract pixel intensity values, calculate intensity ratios (ciliary body/sclera), and then perform correlation analyses and test associations with calculated uveoscleral outflow (see FIGS. 2A-2G). In further aspects, machine learning and deep learning may be employed to automate the segmentation step. Differential signals among the ROIs will indicate relative amounts of uveoscleral flow and will be associated with low versus large IOP fluctuation.

### EXAMPLES

As shown in Table 2, below, three glaucoma patients were imaged at the baseline and two of them (#1 and #3) were imaged again one week after daily nighttime treatment with latanoprost 0.005%. The ratios between the average pixel intensity in the ciliary body and the average pixel intensity in the anterior sclera under baseline conditions were 0.78, 0.78, and 0.85. With latanoprost treatment, the IOP reduction was 20% and 24% for subjects #1 and #3. Repeat UBM imaging and analysis showed an increase in the pixel intensity ratio after treatment, but use of data in the potential subscleral space needs to be added to this simple intensity ratio model. The intensity gradient plot along the central A-line observes a dip in the curve indicating the uveoscleral outflow.

TABLE 2

| | Baseline | | | | Treatment with Latanoprost | | | |
|---|---|---|---|---|---|---|---|---|
| | | Ciliary | | | | Ciliary | | |
| | Sclera | Body | Intensity | | Sclera | Body | Intensity | |
| Subject | Intensity | Intensity | Ratio | IOPcc | Intensity | Intensity | Ratio | IOPcc |
| #1 (65 | 142.80 | 110.75 | 0.78 | 17.5 | 142.92 | 116.35 | 0.81 | 14 |
| years) | | | | | | | | |
| #2 (58 | 147.14 | 115.09 | 0.78 | 19.7 | NA | NA | NA | NA |
| years) | | | | | | | | |
| #3 (61 | 140.57 | 118.86 | 0.85 | 23.3 | 135.34 | 129.54 | 0.96 | 17.6 |
| years) | | | | | | | | |

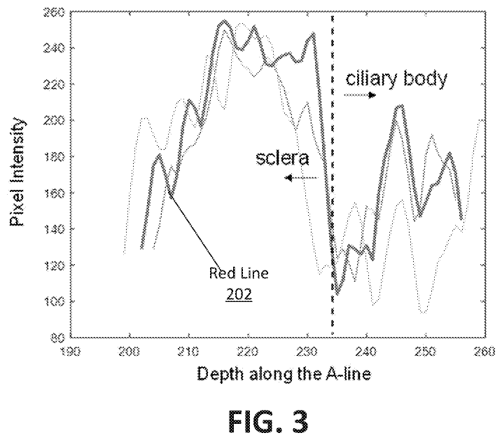

Referring to FIG. 3, there is a dip in intensity in the transition zone from the sclera to the ciliary body as noted by the vertical dashed line. The red line 202 is for the intensity distribution along the central A-line (the center of the whole region of interest), and the other two thinner lines are for the A-lines 10 pixels away from the central A-line on both sides that show a similar pattern as the central one. After a larger database of UBM images is acquired and analysis of the ROI is performed as represented in FIG. 3, then uveoscleral outflow (Fu) will be initially qualitatively characterized in tertiles relative to the IOPs of the cohort composed of patients and controls with healthy eyes. Progress toward this database of patients and controls is ongoing and a priority. This future pixel intensity data will be used initially to qualitatively low-medium-high Fu based on signal intensity. After establishing a larger dataset of AHD data controls and patients, then IOP and outflow facility will be modeled using the measured variables of the modified Goldmann equation.

It is to be appreciated that all or some the above described steps can be performed by computer-readable instructions executed by a processor or one or more processors. As used herein, a processor is a physical, tangible device used to execute computer-readable instructions. Furthermore, “processor,” as used herein, may be used to refer to a single processor, or it may be used to refer to one or more processors. For example, the processor(s) of the remote or mobile device described herein may perform some of the steps disclosed.

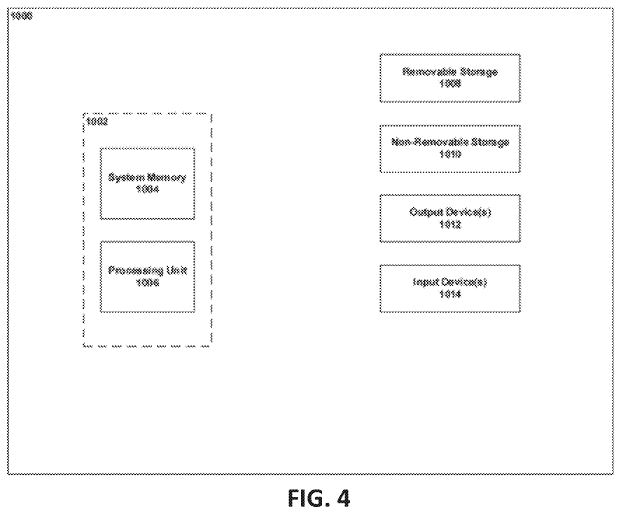

When the logical operations described herein are implemented in software, the process may be executed on any type of computing architecture or platform. For example, referring to FIG. 4, an example computing device upon which embodiments of the invention may be implemented is illustrated. In particular, at least one processing device described above may be a computing device, such as computing device 1000 shown in FIG. 4. For example, computing device 1000 may be a component of a cloud computing and storage system. Computing device 1000 may comprise all or a portion of a server, as described herein. Furthermore, computing device 1000 may comprise all or a portion of the clients (e.g., person electronics devices, smart phone, tablet, personal computer, laptop computer, etc.), as described herein. The computing device 1000 may include a bus or other communication mechanism for communicating information among various components of the computing device 1000. In its most basic configuration, computing device 1000 typically includes at least one processing unit 1006 and system memory 1004. Depending on the exact configuration and type of computing device, system memory 1004 may be volatile (such as random access memory (RAM)), non-volatile (such as read-only memory (ROM), flash memory, etc.), or some combination of the two. This most basic configuration is illustrated in FIG. 4 by dashed line 1002. The processing unit 1006 may be a standard programmable processor that performs arithmetic and logic operations necessary for operation of the computing device 1000.

Computing device 1000 may have additional features/functionality. For example, computing device 1000 may include additional storage such as removable storage 1008 and non-removable storage 1010 including, but not limited to, magnetic or optical disks or tapes. Computing device 1000 may also contain network connection(s) 1016 that allow the device to communicate with other devices. Computing device 1000 may also have input device(s) 1014 such as a keyboard, mouse, touch screen, scanner, etc. Output device(s) 1012 such as a display, speakers, printer, etc. may also be included. The additional devices may be connected to the bus in order to facilitate communication of data among the components of the computing device 1000. All these devices are well known in the art and need not be discussed at length here. Though not shown in FIG. 4, in some instances computing device 1000 includes an interface. The interface may include one or more components configured to transmit and receive data via a communication network, such as the Internet, Ethernet, a local area network, a wide-area network, a workstation peer-to-peer network, a direct link network, a wireless network (e.g., Bluetooth), or any other suitable communication platform. For example, interface may include one or more modulators, demodulators, multiplexers, demultiplexers, network communication devices, wireless devices, antennas, modems, and any other type of device configured to enable data communication via a communication network. Interface may also allow the computing device to connect with and communicate with an input or an output peripheral device such as a scanner, printer, and the like.

The processing unit 1006 may be configured to execute program code encoded in tangible, computer-readable media. Computer-readable media refers to any media that is capable of providing data that causes the computing device 1000 (i.e., a machine) to operate in a particular fashion. Various computer-readable media may be utilized to provide instructions to the processing unit 1006 for execution. Common forms of computer-readable media include, for example, magnetic media, optical media, physical media, memory chips or cartridges, a carrier wave, or any other medium from which a computer can read. Example computer-readable media may include, but is not limited to, volatile media, non-volatile media and transmission media. Volatile and non-volatile media may be implemented in any method or technology for storage of information such as computer readable instructions, data structures, program modules or other data and common forms are discussed in detail below. Transmission media may include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1006 may execute program code stored in the system memory 1004. For example, the bus may carry data to the system memory 1004, from which the processing unit 1006 receives and executes instructions. The data received by the system memory 1004 may optionally be stored on the removable storage 1008 or the non-removable storage 1010 before or after execution by the processing unit 1006.

Computing device 1000 typically includes a variety of computer-readable media. Computer-readable media can be any available media that can be accessed by device 1000 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 1004, removable storage 1008, and non-removable storage 1010 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1000. Any such computer storage media may be part of computing device 1000.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby fully incorporated by reference into this application in order to more fully describe the state of the art to which the methods and systems pertain.

The following publications are expressly incorporated by reference and made a part hereof:
Allingham, R. R., K. F. Damji, S. E. Moroi, D. J. Rhee, S. F. Freedman, S. G. Asrani, and C. C. Teng. 2020. Shields Textbook of Glaucoma (Wolters Kluwer: Philadelphia, PA).
Boese, E. A., C. Clark, and S. E. Moroi. 2018. “Plateau iris configuration as a possible structural risk factor for drug-induced ciliochoroidal effusion mechanism of acute angle-closure crisis.” In American Glaucoma Society Annual Meeting. New York City, NY.
Bourne, R. R., H. R. Taylor, S. R. Flaxman, J. Keeffe, J. Leasher, K. Naidoo, K. Pesudovs, R. A. White, T. Y. Wong, S. Resnikoff, J. B. Jonas, and Study Vision Loss Expert Group of the Global Burden of Disease. 2016. ‘Number of People Blind or Visually Impaired by Glaucoma Worldwide and in World Regions 1990-2010: A Meta-Analysis’, PLOS One, 11: e0162229.
Brubaker, R. F. 2001. ‘Measurement of uveoscleral outflow in humans’, J Glaucoma, 10: S45-8.
De Moraes, C. G., J. M. Liebmann, and L. A. Levin. 2017. ‘Detection and measurement of clinically meaningful visual field progression in clinical trials for glaucoma’, Prog Retin Eye Res, 56:107-47.
Garza, P. S., X. Man, J. H. Queen, B. M. Ayres, T. Mcclendon, E. A. Parrish, D. M. Reed, and S. E. Moroi. 2020. ‘Size Matters for Interplicata Diameter: A Case-Control Study of Plateau Iris’, Ophthalmol Glaucoma, 3:475-80.
Ismail, R., A. Azuara-Blanco, and C. R. Ramsay. 2014. ‘Variation of clinical outcomes used in glaucoma randomised controlled trials: a systematic review’, Br J Ophthalmol, 98:464-8.
Man, X., R. Costa, B. M. Ayres, and S. E. Moroi. 2016. ‘Acetazolamide-induced bilateral ciliochoroidal effusion syndrome in plateau iris configuration’, American Journal of Ophthalmology Case Reports, 3:14-17.
Sagara, T., D. D. Gaton, J. D. Lindsey, B. T. Gabelt, P. L. Kaufman, and R. N. Weinreb. 1999. ‘Topical prostaglandin F2alpha treatment reduces collagen types I, III, and IV in the monkey uveoscleral outflow pathway’, Arch Ophthalmol, 117:794-801.
Toris, C. B., M. Gagrani, and D. Ghate. 2021. ‘Current methods and new approaches to assess aqueous humor dynamics’, Expert Review of Ophthalmology, 16:139-60.
Zhang, L., C. T. Hood, J. P. Vrabec, A. L. Cullen, E. A. Parrish, and S. E. Moroi. 2014. ‘Mechanisms for in-the-bag uveitis-glaucoma-hyphema syndrome’, J Cataract Refract Surg, 40:490-2.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

Expressly incorporated by reference and made a part hereof is the publication by inventors of this invention, Yanhui Ma; Sunny Kwok; Jun Liu; Sayoko E Moroi “Characterizing uveoscleral outflow from high-frequency ultrasound imaging in humans,” Investigative Ophthalmology & Visual Science June 2024, Vol. 65, 5143. doi:, which was published less than one year prior to the filing date of the provisional application to which this application claims priority.

What is claimed is:

1. A method of characterizing uveoscleral flow (Fu) from ultrasound biomicroscopy (UBM) by analyzing pixel intensity distributions in the anterior perilimbal sclera and ciliary body:
capture, using a UBM protocol, longitudinal images of a patient's eye at nasal and temporal meridians with an ultrasound probe, wherein ultrasound scans are obtained at Longitudinal Nasal, pointing temporal; and Longitudinal Temporal, pointing nasal;
segmenting an anterior sclera and ciliary body of the patient's eye using the captured images from the ultrasound scans to determine one or more regions of interest (ROI);
analyzing a pixel intensity gradient along multiple vertical A-lines evenly across the ROI to delineate the intensity changes at a transition band from the sclera to the ciliary body, wherein average pixel intensity values within the ciliary body and anterior sclera regions are assessed, and a ratio between them is calculated, wherein the intensity ratio is calculated as one of the metrics for association analysis with uveoscleral outflow; and
determining the Fu by averaging intensity profiles of the multiple vertical A-lines, plotting an intensity gradient plot derived from the average intensity profiles, and observing a dip in the intensity gradient plot, which indicates the uveoscleral outflow.

2. The method of claim 1, wherein the captured images are aligned for consistent measurement.

3. The method of claim 2, wherein reference lines in the captured images are used for alignment.

4. The method of claim 3, wherein software executing an algorithm on a computing device receives the captured images and the software detects any tilt in the angles of the reference marks and rotates the image to correct orientation.

5. The method of claim 1, wherein the captured images comprise B-mode images.

6. The method of claim 5, wherein captured images of anterior sclera and ciliary body are manually segmented relative to a limbus.

7. The method of claim 6, wherein pixel intensity gradient along the central A-line is analyzed to delineate intensity changes at a transition band from sclera-to-potential subscleral space-to-ciliary body using software algorithms executing on the computing device.

8. The method of claim 7, wherein the software algorithms comprise the steps of:
marking a location of a limbus and a distance posterior of the limbus (toward an anterior sclera) as boundaries for the ROI;
tracing an interface between the anterior sclera and the ciliary body;
an anterior surface of the anterior sclera is traced to connect the ROI boundaries and the anterior scleral-to-potential subscleral space-to-ciliary body interface for the segmentation of the anterior sclera;
a posterior surface of the ciliary body is traced to connect the ROI boundaries and the anterior scleral-to-potential subscleral space-to-ciliary body interface for the segmentation of the ciliary body, wherein the central A-line is defined as the A-line passing the centroid (or geometric center) of the selected anterior sclera and ciliary body region;
average pixel intensity values within the ciliary body and anterior sclera regions are assessed, and the ratio between them (i.e., ciliary body/anterior sclera) is calculated,
wherein the pixel intensity of the sclera is relatively high due to the compact nature of this tissue and relatively low aqueous humor content,
wherein pixel intensity of the ciliary body is more variable due to the nature of the complex tissues and the relative amount of aqueous humor in the tissue as part of the Fu pathway represented in a modified Goldmann equation, and
wherein relative differences in pixel intensity can be detected in the ROI under baseline conditions that demonstrate a trend with IOP with a higher signal intensity ratio indicating a higher IOP under baseline conditions.

9. The method of claim 8, wherein two vertical lines mark a location of the limbus and 4 mm posterior from the limbus (toward the anterior sclera) are drawn as the boundaries for the regions of interest (ROI), wherein the interface between the anterior sclera and ciliary body is manually traced first, the anterior surface of the anterior sclera is then traced to connect the ROI boundaries and the anterior scleral-to-potential subscleral space-to-ciliary body interface for the segmentation of the anterior sclera, the posterior surface of the ciliary body is traced to connect the ROI boundaries and the anterior scleral-to-potential subscleral space-to-ciliary body interface for the segmentation of the ciliary body.

10. The method of claim 9 wherein the multiple vertical A-lines are defined as A-lines passing through a centroid (or geometric center) of the selected anterior sclera and ciliary body region.

11. The method of claim 10, wherein average pixel intensity values within the ciliary body and anterior sclera regions are assessed, and a ratio between them (i.e., ciliary body/anterior sclera) is calculated, wherein a pixel intensity of the sclera is relatively high due to the compact nature of the tissue and relatively low aqueous humor content, and the pixel intensity of the ciliary body is more variable due to the nature of the complex tissues and the relative amount of aqueous humor in the tissue as part of the Fu pathway represented in the modified Goldmann equation.

12. The method of claim 11, wherein relative differences in pixel intensity can be detected in the ROI under baseline conditions that demonstrate a trend with IOP with a higher signal intensity ratio indicating a higher IOP under baseline conditions.

13. The method of claim 1, wherein the longitudinal images of a patient's eye at nasal and temporal meridians are acquired using ahigh-frequency ultrasound probe.

14. The method of claim 13, wherein the high-frequency ultrasound probe has a frequency in a range of 20 to 50 Mhz.

15. The method of claim 13, wherein four scans are obtained at the 3 and 9 o'clock meridians of the patient's eye.

16. The method of claim 13, wherein the ultrasound scans are obtained in video mode.

17. The method of claim 1, wherein the anterior sclera and ciliary body are manually segmented.

18. The method of claim 7, wherein the modified Goldmann equation comprises:
$$IOP = \frac{AHF - Fu}{C t r a b} + Pe .$$
